(19) United States
(12) Patent Application Publication
(10) Pub. No.: US 2020/0260981 A1
(43) Pub. Date: Aug. 20, 2020

(54) IMPLANTABLE MEDICAL APPARATUS WITH INTERCONNECTED ELECTRODES

(71) Applicant: NOVELA NEUROTECHNOLOGIES INC., Alameda, CA (US)

(72) Inventors: Peter MANKOWSKI, Alameda, CA (US); Salam GABRAN, Toronto (CA)

(21) Appl. No.: 16/791,438

(22) Filed: Feb. 14, 2020

Related U.S. Application Data
(60) Provisional application No. 62/806,348, filed on Feb. 15, 2019.

(51) Int. Cl. A61B 5/0478; A61N 1/05; A61B 5/00; A61N 1/372

(52) U.S. Cl. CPC A61B 5/0478; A61N 1/0529; A61B 2562/225; A61N 1/37229; A61B 5/002

(57) ABSTRACT

A module of implantable cortical and/or intra-cortical comprising a first electrode and a second electrode. Each electrode has a base with interconnect pads adapted to be connected to a processor, a stem having interface pads on a first face, the interface pads adapted to contact soft tissue, routing tracks between the interconnect pads and the interface pads, and abutment surfaces on the second face of each said electrode. The abutment surfaces of the first electrode and of the second electrode are complementary, whereby the first electrode and the second electrode form a translation joint along a length of the stem when the electrodes are joined.

### FIELD OF THE DISCLOSURE

The present disclosure relates generally to medical implantable cortical and/or intra-cortical electrodes developed for interfacing neurons and providing multichannel stimulation and/or recording.

### DESCRIPTION OF THE RELATED ART

Cortical recording and functional electrical stimulation (FES) are emerging therapeutic techniques employed in developing electrotherapeutic techniques for treating nervous system disorders. Functional electrical stimulation of the central nervous system (CNS) is based on delivering a stimulating electric charge to the targeted brain structure through electrodes implanted in the brain cortex. These evolving electrotherapeutic techniques provide therapies for several medical conditions complementary to regular surgical and chemical therapies. Also, recording neural electrical activities may be used for studying population dynamics and information coding across neuron ensembles.

The emerging neuro-interfacing technology introduces many challenges. The acceptance of new therapeutic procedures implementing neuro-modulation is challenged by several factors directly linked to the fact that those are limited to a narrow field of view, notably because of the manufacturing process. Traditionally, the active scanning site consists of all active components and the opposite ‘blank’ surface performs no scanning at all. The number of available channels per electrode in known technologies is limited by factors such as electrode dimensions and fabrication technology.

### SUMMARY OF THE INVENTION

In accordance with the present disclosure, there is provided a module of implantable cortical and/or intra-cortical comprising: a first electrode and a second electrode, each said electrode having a base with interconnect pads adapted to be connected to a processor, a stem having interface pads on a first face, the interface pads adapted to contact soft tissue, routing tracks between the interconnect pads and the interface pads, and abutment surfaces on the second face of each said electrode; wherein the abutment surfaces of the first electrode and of the second electrode are complementary, whereby the first electrode and the second electrode form a translation joint along a length of the stem when the electrodes are joined.

In accordance with another aspect, for instance, multi-layer cortical or intra-cortical electrode that may provide neuro-interfacing through stimulation and/or recording.

In accordance with another aspect, for instance, the electrode may have one or more shafts according to the design requirements and application.

In accordance with another aspect, for instance, Each shaft may comprise a group of stimulation and/or recording pads and routing tracks. The electrodes may comprise both stimulation and/or recording electrodes for enhanced functionality.

In accordance with another aspect, for instance, the number, dimensions and/or layout of pads may vary according to the design requirements and application.

In accordance with another aspect, for instance, each probe's shaft consists of a metal plate/sheet to shape and control energy distribution.

In accordance with another aspect, for instance, each metal plate can be used in one of three modes; floating, GND connected, VCC connected.

In accordance with another aspect, for instance, the electrode may comprise interconnect pads to provide connection to external circuits and/or or devices.

In accordance with another aspect, for instance, the electrode layout may have tapered shaft tip to facilitate penetration and/or improve the structural rigidity and mechanical stability during insertion. The tapered tip may be designed to distribute the tip forces uniformly and evenly during electrode insertion across the shaft to enhance the structural failure, and/or maintain the targeted insertion path.

In accordance with another aspect, for instance, the electrode may be made of flexible and/or rigid materials.

In accordance with another aspect, for instance, the electrode structure may comprise a rigid structural layer and additional layers formed on top of the structural layer.

In accordance with another aspect, for instance, the electrode structure may provide the required mechanical strength and/or flexibility to support the electrode during insertion and during operation. The electrode design may provide the required axial rigidity and lateral flexibility to support the electrode during insertion and operation.

In accordance with another aspect, for instance, the electrode shaft may have appropriate width to provide mechanical strength, minimize tissue trauma and displacement as well as accommodate the stimulation and/or recording pads.

In accordance with another aspect, for instance, each electrode shaft may have appropriate length to penetrate deep enough to come in contact with the targeted neurons.

In accordance with another aspect, for instance, the width of routing tracks may be appropriate to provide reliable electrical connections.

In accordance with another aspect, for instance, the stimulation pads may be of appropriate area to deliver the stimulation charge.

In accordance with another aspect, for instance, the stimulation pads have larger area than recording pads that detect neuron spikes, however the stimulation pads may be used to detect local field potentials.

In accordance with another aspect, for instance, there may be one or more reference pads and may have optimized and/or different design according to design requirements.

In accordance with another aspect, for instance, recording pads may be of appropriate area to detect neuron spike.

In accordance with another aspect, for instance, the recording pads may have a polygonal or circular footprint with dimensions suitable to pick up the required signal.

In accordance with another aspect, for instance, the electrode may have one or more metal or conductive layers that may be connected or biased or electrically floating according to the design requirements.

In accordance with another aspect, for instance, the layout, number and area of contact, stimulation and/or recording pads per each composite layer may vary independent of the other composite layer may there exist more than a single composite layer according to the design requirements and application.

In accordance with another aspect, for instance, the interconnect pads may have appropriate dimensions to provide reliable connection to interconnect wires

In accordance with another aspect, for instance, the interconnect pads on the electrode base may have dimensions and layouts that satisfy the design requirements and may be designed to match the footprint of commercial, custom and/or standard connectors.

In accordance with another aspect, for instance, the interconnect pads may have a polygonal footprint with dimensions suitable to provide reliable contact with the interconnect wire.

In accordance with another aspect, for instance, the electrode may be implanted directly without using implantation devices.

In accordance with another aspect, for instance, the electrode may be used for short term or extended durations.

In accordance with another aspect, for instance, the layout and dimensions of the carrier wafer used for stacking the electrodes into multi-dimensional may vary according to the application and design requirements.

In accordance with another aspect, for instance, the device may be made of 2 separate single device that may be attached back-to-back to provide contact with targets at both sides of the device.

In accordance with another aspect, for instance, in case the device may be created by attaching 2 separate single devices attached back-to-back, an intermediate metallic layer or 2 intermediate metallic layers may be formed between the 2 separate single devices. The single or double metallic layers may provide the propagation of stimulation fields to undesired locations within the tissue.

In accordance with another aspect, for instance, in case of two electrodes used as one assembly, they will always be in the-back-to-back configuration with sensing elements directed toward opposite directions as per each sub-probe element.

In accordance with another aspect, for instance, one or more electrodes may be stacked to create multi-dimensions array. The number of planar electrodes per array may vary according to the design requirements and application.

In accordance with another aspect, for instance, one or multiple guiding rails are used to prevent sideways sliding. Only sliding in the vertical direction is desirable for that assembly.

In accordance with another aspect, for instance, a mounting hole on multiple are used to secure both probes and prevent shifting during operations.

In accordance with another aspect, for instance, securing offset settings uses one or multiple screws. Inner surface of each probe will always match the opposite probe's inner area, providing sliding surface and alignment among rails.

In accordance with another aspect, for instance, mounting screws used to secure the current probe offset can be replaced, moved, removed and alter based on current design requirements.

In accordance with another aspect, for instance, relative offset between each probe can be managed before during or after device insertion.

In accordance with another aspect, for instance, There is not final offset that will permanently lock both probes as screws are exposed and available to tuning at any time.

In accordance with another aspect, for instance, variations of offset between probes can be used for calibration purposes to maximize accuracy of performance based on current requirements.

In accordance with another aspect, for instance, calibration or enabling tow probes at the same time can be used for simultaneous reading or stimulation at higher sensitivity and granularity than other methods.

In accordance with another aspect, for instance, each probe's metal sheet at the back influences signal radiation patterns, lobe's shape and other signal properties.

In accordance with another aspect, for instance, probe facing up and the corresponding probe facing the opposite direction can distribute radiation patterns covering all of 360 degrees or 180 degrees if required.

In accordance with another aspect, for instance, probe's offset during initial insertion or device usage can lead to locating the best placement allowing increased sensitivity of reading and stimulation effectiveness.

In accordance with another aspect, for instance, reading both probes and moving the offset between them at the same time can be used to model 3D depth perception and additional functions not visible by other methods.

In accordance with another aspect, for instance, in the case of one probe malfunction, the 2^{nd} unit will provide a back-up scanning and stimulation, which is an important element of patient's safety and FDA requirements as per any medical devices.

### BRIEF DESCRIPTION OF THE DRAWINGS

FIG. 1 is a schematic top view of an electrode used in a module of electrodes in accordance with an embodiment of the present disclosure;

FIG. 1.1 is a schematic view showing a top and bottoms of the electrode of FIG. 1;

FIG. 2 is a schematic top view of an exemplary layout showing hidden tracks which are inside the electrode and not exposed at the electrode surface;

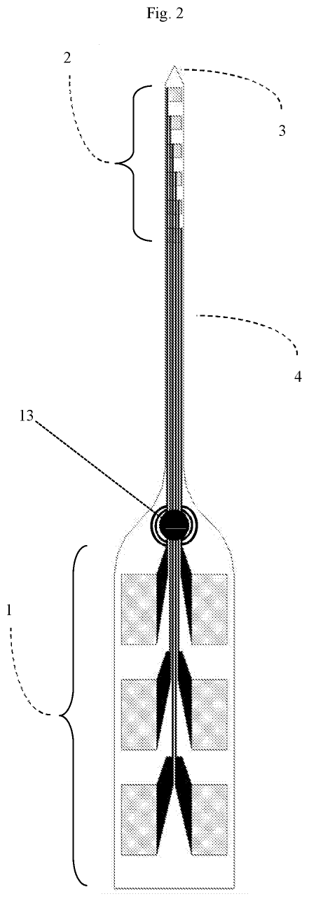

FIGS. 3A-3D are schematic sectional views of exemplary complementary configurations of stems of pairs of the electrode of FIG. 1, in a module;

FIG. 4 is a schematic side view of part of the electrode showing several layers;

FIGS. 5A and 5B are schematic sectional views showing fasteners for the configurations of FIGS. 3A-3D;

FIG. 6 is a schematic view showing an assembled multi-dimensional array with the electrodes of FIG. 1, with different or no offsets;

FIG. 7 is a picture of an assembled multi-dimensional array comprising two planar electrodes; and

FIG. 8 is a perspective view of two planar electrodes forming a module, with zero relative offset and matching metal sheets backing each other as one assembly option.

### DETAILED DESCRIPTION OF THE INVENTION

The introduced design methodology and architecture, and the presented cortical and/or intra-cortical electrodes take into consideration biocompatibility and functionality for the electrodes and/or ability to assemble matching units back-to-back. The presented electrode architecture and design methodology may increase the number of interfacing stimulation and/or recording channels on the electrode shaft while vis à vis the shaft dimensions. The increased number of available channels may assist in electrode functionality, and a reduced footprint may contribute to its biocompatibility. The improved biocompatibility may be achieved by the electrode structural design, layout and/or dimensions. The electrode architecture may include multiple metallization, dielectrics and passivation layers. The electrode layout and dimensions may contribute to enhancing the electrode biocompatibility by reducing tissue trauma and displacement. This may lead to minimizing the intensity of the immune system foreign body response.

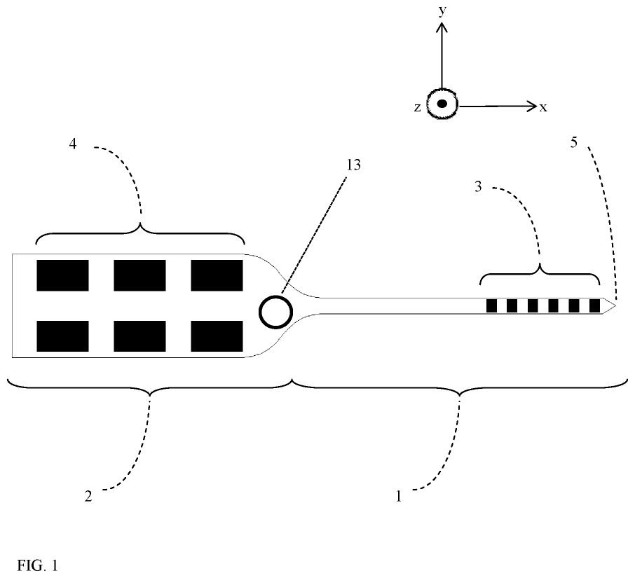

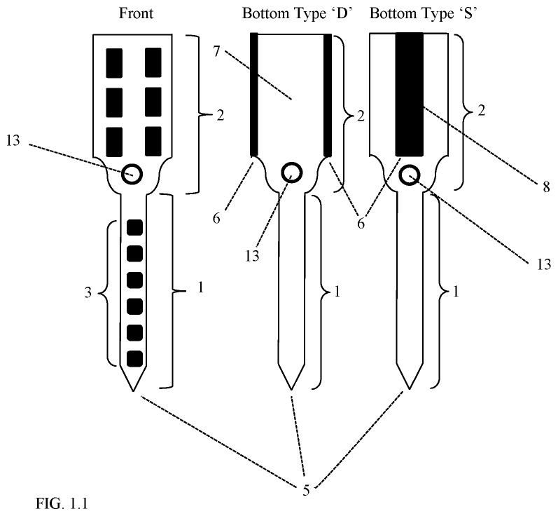

Referring to the drawings and more particularly to FIG. 1, there is shown an embodiment of the electrode, which may also be referred to as a probe. The electrode may have several regions, such as an electrode stem featuring one or more shafts 1, an electrode base 2, interface stimulation and/or recording pads 3, interconnect pads 4, an electrode tip 5 (e.g., such as a tapered tip), routing tracks 6 (FIG. 1.1), a mounting hole 13. The interconnect pads 4 may couple the electrode to external circuits and devices, while the interface stimulation and/or recording pads 3 may connect the electrode to the tissue to deliver charge and/or record signals and/or neuron activities. The associated pairs of interconnect pad 4 and interface pad 3 may be connected together using the routing tracks 6.

In conventional electrode designs, all the metallic components including routing tracks, interconnect pads and interface stimulation and/or recording pads are accommodated within the same metallization layer. As a result, the electrode shaft 1 has to accommodate the routing tracks 6 and interface pads 3. The routing tracks 6 share the available shaft area with the interconnect pads 3. The routing tracks may be considered an overhead which consumes shaft space and limit the number of available channels per shaft. In the present disclosure, the metallic components may be distributed among more than one metallization layer. Associated metal components may be connected through vias, i.e. small openings in the metallization layer(s) that allows a conductive connection between different layers.

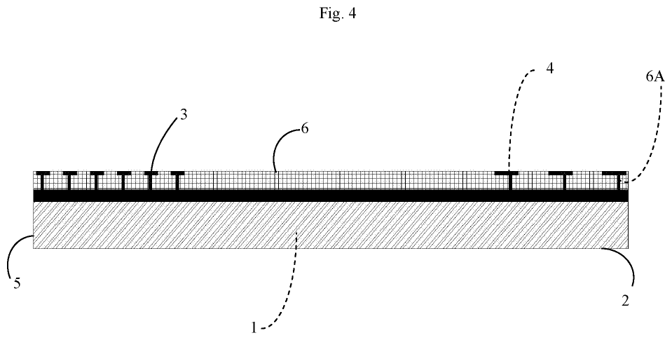

FIG. 1 shows an exemplary top view sketch of the electrode to illustrate a contemplated electrode contour, with a tapering of the electrode base 2 leading to the electrode shaft 1. The electrode base 2 supports the interconnect pads 4, whereas the electrode shaft 1 supports the stimulation and/or recording pads 3. In FIG. 1.1, the electrode tip 5 is magnified to show some of the tracks 6, vias 6A and stimulation and/or recording pads 3. The number of pads 3,4, layout of pads and dimensions and layout of the routing tracks 6 may vary according to the required electrode design and dimensions. A side longitudinal cross-section view of the electrode is presented in FIG. 4. It shows tracks 6, interconnect pads 4, stimulation and/or recording pads 3, passivation and insulation layers, vias, and an electrode structural layer. A dielectric thin film provides electrical insulation between the metallization layers. Connection is established between the tracks 6 and associated pads 4 through the dielectric layer using vias. The topmost metallization layer, shown by way of the pads 3 and 4, may be covered with a biocompatible dielectric passivation layer to protect the electrode surface implanted within the tissue. The dielectric layers including the passivation layer may have openings to expose the stimulation and/or recording pads 3 as well as the interconnect pads 4.

The region of the electrode identified as the shaft 1 may have a layout with a tapered tip 5 to facilitate tissue penetration. The tapered tip 5 may be designed to distribute the tip forces uniformly and evenly across the shaft during electrode insertion to enhance the structural resistance to failure, as well as maintain the targeted insertion path, and/or may be designed to facilitate tissue penetration.

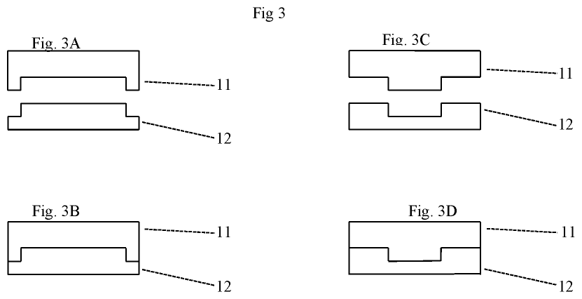

The electrode width may have any appropriate size to accommodate the required number of stimulation and/or recording pads 3 and the routing tracks 6. For example, the width of the electrode shaft may range from 60 to 250 μm. The electrodes may have one or more shafts 1 of equal or different lengths according to the design requirements and application. The electrode may be of any appropriate length to satisfy the design requirements and application. The electrode may have some or all of the components identified previously, for example as in FIGS. 1, 1.1 and 4, such as one or more electrode shafts 1 on the electrode stem and/or electrode base 2. The electrode may be used as planar electrode or may be stacked into multi-dimensional array as shown in FIG. 3. The electrode may be stacked into multi-dimensional array using different methods including but not limited stacking wafers, stacking assemblies back to back, using one or multiple rails for stability and offset adjustments as shown in FIG. 3. The stacking wafers may be of any dimension and material that satisfies the design requirements and application. Hence, the metal layers featuring the routing tracks may be embedded in passivation and/or dielectric material.

The stimulation and/or recording pads 3 may have a polygonal or circular footprint with dimensions suitable to deliver the required charge, though other shapes are possible. For example, a range of dimensions for the stimulation and/or recording pads 3 may range from 10 microns to 110 microns. The interconnect pads 4 may have a polygonal footprint, or other non-polygonal shapes, with dimensions suitable to provide reliable contact with an interconnect wire. For instance, a range of dimensions for the interconnect pads 4 may range from 100 microns to 500 microns. The interconnect pads 4 on the electrode base 2 may have dimensions and layouts that satisfy the design requirements and may be designed to match the footprint of commercial, custom and/or standard connectors. One of the interconnect pads 4 may be a reference pad that may be designed to have different size or layout as required. In terms of electric connections, the electrode can for example be used for differential operation or single-ended operation. In the case of single-ended operation, one of the pads may be used as a reference for all the channels, i.e., a reference pad. This reference pad can be arbitrarily specified by the end user during device operation. In some cases, the reference pad can be specified during electrode fabrication as a specific product and may have different dimensions compared to the regular pads.

The electrode may be implemented on brittle, rigid, and/or flexible substrates. The structural layer may be made of but not limited to silicon of any crystal orientation, metal and/or polyimide. The dielectric and passivation materials may be biocompatible and may be made of but not limited to silicon-oxide, silicon-nitride, parlin, polyurethane and/or polyimide. The metallization materials used in the electrode may be biocompatible and may be made of but not limited to chrome, gold, platinum, iridium and/or titanium or their compounds. The metal layers and the electrode surface may be treated or post processed to improve the characteristics of the electrode or satisfy the design constraints. Pad post processing may be done through several techniques including but not limited to depositing a rough layer to increase the pad surface area and the electrode-electrolyte interface area, and this may be done using but not limited to electroplating or electroless-plating. This may apply to either or both of the pads 3 and 4.

The electrode may include strain relief elements to minimize transferring mechanical forces from the electrode base 2 to its shafts 1. These forces may result when the electrode base 2 is disturbed and may disrupt the stability of the implanted stem, the stability of the channel-tissue interface, and/or may cause artifacts including but not limited to displacement artifacts. That challenge may be solved by providing an assembly of two matching electrodes back-to-back, for support, stability, strain relief and adjustments of area of measurement.

The electrode may have a modular design which includes mounting hole/holes 13 or attaching the electrode to another device which may be a printed circuit board of any design. The printed circuit board may be designed to be populated with any device that may be connected to the electrode; this may include pin header or connectors, or other devices that would satisfy the design requirements. The electrode shown in FIG. 1 may be of the type used in a back to back arrangement, and thus may be part of dual assembly electrodes. Hence, a module may consist of an assembly of two back-to-back electrodes (such as the one of FIG. 1) guided by one or multiple rails for control. The rails form a mechanical interface that may block any side displacement of an electrode relative to another during insertion or testing procedures. Stated differently, by way of the rails, the stems of an assembly of electrodes remain longitudinally aligned.

Referring to FIGS. 3A-3D, two schematic sectional views are shown to show contemplated back to back assembly of electrodes with rails. The sectional views show a cross-section of the stems of electrodes 11 and 12. According to an embodiment of FIGS. 3A and 3B, one of the electrodes, shown as 11, has two parallel sliding rails, while a second one of the electrodes 12 has a pair of shoulders. Stated differently, the electrode 11 has a ‘U’ shape, while the electrode 12 is a “negative” of the shape of the electrode 12, for complementary engagement. By the illustrated embodiment, the electrodes 11 and 12 are blocked from moving in a lateral direction Y, but may slide along longitudinal direction X. Electrodes 11 and 12 become a module, set or assembly with a self-guiding mechanism by only allowing motion in two directions (including direction Z). Edges of electrodes 11 and 12 may match both dimensions and corner to corner distance. FIG. 3B shows the final placement of electrodes 11 and 12 while together, with the possibility of sliding in a directional manner along longitudinal direction X, limited by shape's characteristics.

In another embodiment, shown in FIGS. 3C and 3D, only one sliding rail, is used, in electrode 11 (though it could be said that electrode 12 has two parallel rails). The difference between the embodiments of FIGS. 3A-3B and FIGS. 3C-3D is the number of rails, if a rail is defined as having a maximum width. In the embodiment of FIGS. 3C-3D, the rail may be in the middle of electrode 11, though it may be off centered as well. In both these embodiments, there are two pairs of opposing lateral abutment surfaces blocking movement in direction Y. The number of pads 3,4 and the dimensions of pads 3,4 may vary according to the design requirements. The layout and shape of the electrodes 11 and 12 may differ according to the required electrode design and dimensions.

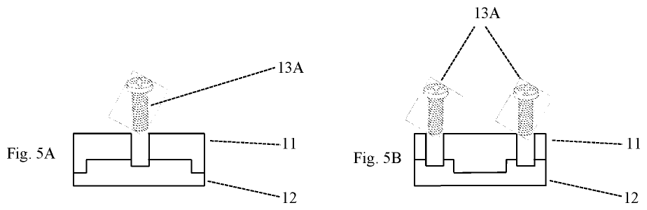

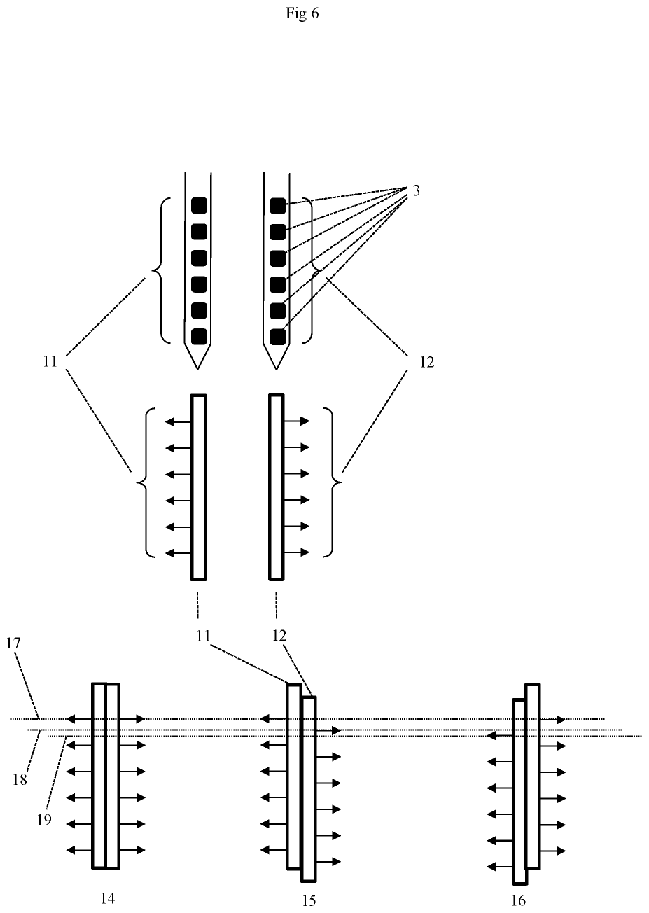

Referring to FIG. 6, the longitudinal sliding capability between two individual probes 11 and 12 may provide an offset between them, as illustrated by 17 and 19, relative to the aligned reference 18. The offset may be a positive or negative with user controlling the amount of displacement and granularity. It is contemplated to secure the electrodes 11 and 12 to block any longitudinal displacement and hence any offset. For example, shown in FIGS. 5A and 5B. the secured may be done by one or multiple mounting holes and fasteners, such as screws 13A, bolts, rivets, welding, soldering, cementing, etc. Each screw 13A may be tightened to lock a user's selection of offset between the electrodes 11 and 12.

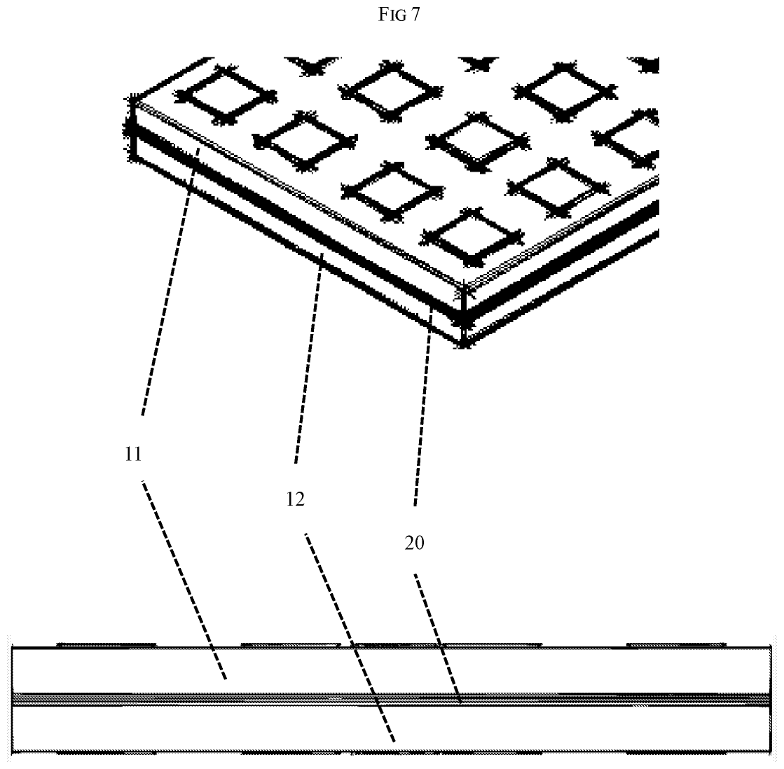

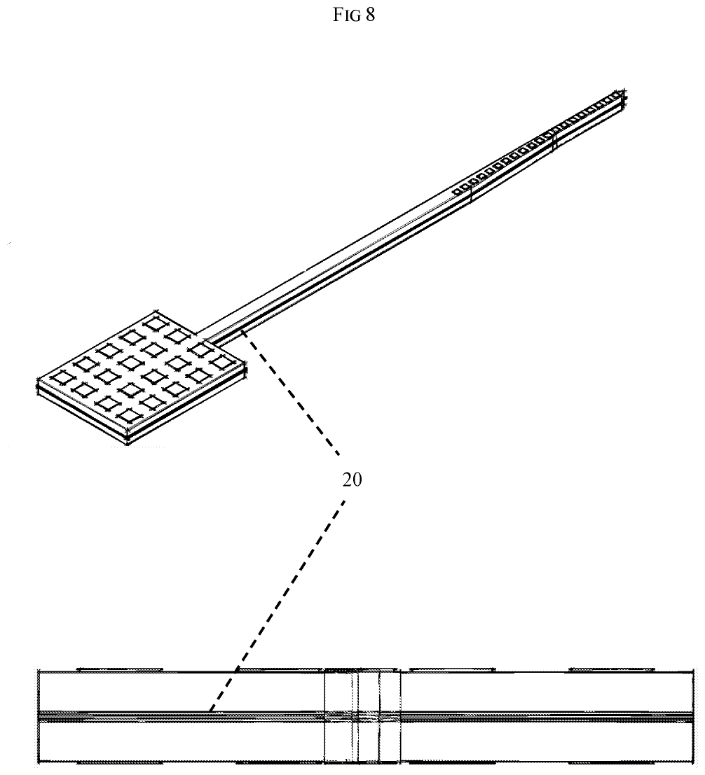

Referring to FIGS. 7 and 8, metal plates 20 may be sandwiched between electrodes 11 and 12, for directional RF lobe and/or energy distribution. The two electrodes 11 and 12 may be capable of directing and adjusting their corresponding energy based on the plates 20, which may deploy existing elements of RF telecommunication—Concentrated Ground Plane Booster Antenna Technology. Each plate 20 could be left in distinct modes, such as unconnected/floating, GND connected, or VCC connected. Each plate 20 could be set up to one of three modes listed above, independently of the opposite side's settings. This energy shaping capability can be used for both the scanning and stimulation activities per each side. Hence, the metal plate backing 20 may provide programmable capabilities to form RF lobes and radiated profiles.

### DETAILED DESCRIPTION OF THE OFFSET SELECTION, LOCKING THE DESIRED SETTING AND MOUNTING SCREW(S) FUNCTIONALITY AND RELEVANCE DURING INSERTION AND ALL MODES OF OPERATION

In accordance with an embodiment of the present disclosure, a process may be used for producing rigid, brittle and/or flexible structures with multiple metallization, dielectric and passivation layers, such as the electrodes described herein. As shown in FIG. 1, in accordance with an embodiment, each electrode may have components and traces on one side of the assembly. FIG. 1 shows regions of the electrode identified as the electrode shaft 1, electrode base 2, stimulation and/or recording pads 3, interconnect pads 4, and the tapered electrode tip 5. The mounting hole 13 may be present for a fastening device such as a screw 13A.

The opposite surface of the electrode may be blank, i.e., with no components. However, the lateral abutment surfaces, such as the one or multiple guiding rails as shown in FIGS. 3A-3D, may be on the opposite surface. The guiding railings may provide ability to slide the electrodes in a longitudinal direction X relative to its matching electrode, i.e., the other electrode in the module, both facing each other as exemplified in FIG. 6 by electrodes 11 and 12. Guiding rail or multiple rails may not lock the electrodes to one another, forming instead a directional guide. Under certain conditions and/or use case those two electrodes can depart and disengage under pressure and tension related to the current use.

In order to keep electrodes 11 and 12 in place along the X and Z directions, a mounting mechanism may be present, as in FIGS. 5A and 5B. In an embodiment, the mounting mechanism consists of the hole 13 or multiples such holes and matching fastener, such as screw 13A.

Referring to FIG. 6, during insertion or any modes of operations, electrodes 11 and 12 need to be adjusted to either no offset between the main planes, as aligned with 18 or any positive offset or negative offset desired per current application, as per 17 or 19. The amount of offset, direction and granularity is user defined and only limited to predictability of sliding probes in accurate and repeatable manner. Configurations 14, 15, and 16 shows samples of offset arrangements that are custom based on measurement's objectives. When a desired offset is attained, e.g., 14, 15, 16, locking screw 13A of the like is tightened to block any sliding or changes in along directions X and Z. Some configurations may require one screw 13, but other configurations may involve more than one screw 13, as in FIG. 5B.

The configurations 14, 15 and/or 16 show two electrodes back-to-back, the electrodes being oriented so as to have the pads 3 facing outwardly, so as to provide the ability to scan outward, on opposite faces. This array of two rows of pads 3 (a.k.a., sensors) facing away from each other opens up another opportunity to improve device operations. Electrodes 11 and probe 12 can correlate each other's readings, level of noise and/or performance by adjusting and comparing incoming readings. For instance, a close-loop method may be used to perform a comparison. Any significant discrepancy between readings (e.g., raw data) from electrodes 11 and 12 may be analyzed to understand the directional nature of the readings from the two electrodes of a module. The configurations 14, 15 and/or 16 can be used to adjust each probe placement to align neuro signal availability to the sensors 3. The alignment may be useful to access neuro nodes. Neuro nodes may produce signals too small to read if the electrode is not close enough. In such a case, unwanted noise may be present, and this may result in an inability to scan a brain's response, affecting the operation of one or both electrodes 11 and 12. The ability to use the module's offsetting capability of the electrodes to pinpoint best depth and regions of scanning may be measured in real time by electronics attached to the electrodes. Once a suitable scan depth is reached, the module may be locked-in by one or several locking screws 13A. The offset can be adjusted during or after the initial insertion by unlocking the screws 13A or like fasteners, and locked back.

Additional functions, setting and ability to manipulate each electrode of a module or the locking mechanism may include:
Releasing, adjusting or automatically changing the electrode's offsets by both manual displacement, for instance using an electrical motor moving either one or both electrodes. Any other means to drive the offset between electrodes 11 and 12 may be used.
Sliding rails can be placed as an array of many parallel shape surfaces. The rails may be treated with substances to improve viscosity to satisfy the design requirements.
Adjustment or control of offset between the electrodes 11, 12 can be automated, i.e., with or without user's control. In an embodiment, no intervention with the device's mechanical or/and software programs may be required, as the automated control may operate some or all decisions.
Tightening screw 13A can be controlled for the locking or/and unlocking functions manually or by electromechanical solutions conducting similar functions.
Accuracy and granularity of the offset between electrodes 11 can be decided based on predefined settings or dynamically as the result of a power-up scanning and decision-making procedure.

A novel feature of the module of electrodes 11 and 12, such as in FIGS. 5A, 5B and 6-8 is the fact that it consists of two electrodes 11 and 12 that are interconnected back to back and move relative to the other in the longitudinal direction X in a controlled, sliding motion. Since each of two electrodes 11 and 12 may be furbished with a sheet of metal covering the back area, e.g., the metal plate 20, the module of electrodes 11 and 12 may have an additional function of being a pseudo-RF transceiver with programmable direction or lack of based on the current set of requirements.

Additionally, the module's depth perception may be varied and adjusted by sliding one electrode relative to the other. By making two electrodes 11 and 12 on rails or like complementary sliding arrangements, while sliding enabled as desired, accuracy and adjustments may be enhanced in neuro scanning characteristics by both depth perception and granularity of measurements on both sides of the electrode assembly, and/or and increase the density of interface channels.

The module described above may feature multi-layer implantable cortical and/or intra-cortical electrodes with the ability of stacking into multi dimension arrays. The electrode design methodology may be based on multiple metallization layers connected by vias. The design methodology may allow an increase in the number of available channels while minimizing the electrode shaft dimensions. The electrodes may be stacked to create multi dimensions arrays. The electrodes, in certain conditions, might have two identical sub-blocks connected together to enable scanning and stimulation on both sides of the electrode's assembly (up direction and down direction). The bottom of each electrode may be a resin filled substrate, to facilitate sliding between two surfaces, such as in the arrangement of electrodes 11 and 12. Matching rails and shoulders and/or slots on the electrodes rear surfaces may provide a safety mechanism required to move electrodes and create an offset per each side.

Another feature is that electrodes 11 and 12 may provide scanning and stimulus in opposite directions. For instance, the module of electrodes 11 and 12 may effectively cover a full 360-degree field of view, while maintaining ability of reading independently as well. This may result in a greater scanning coverage under test and a number of options to offset each electrode. The metal plate 20, in comparison to the popular RF ground plate booster provides a directional lobe shape and radiated power distribution control per each of two probes; simultaneously or independently. A Half-Power beam width (HPWP) per each side may provide the 360 degrees distribution if the metal plates 20 are set up to be both floating.

The fact that the electrodes 11 and/or 12 may have a matching shaped metal RF sheet 20 turns the electrode(s) into an RF transceiver device with lobe directions, radiation pattern and signal propagation efficiencies common among telecommunication equipment. In addition to the metal shield-sheet, three modes of operations and control may be available for selection; floating/not connected to either GND/VCC, ground connected, voltage/current injection at any constant or/and changing parameters settings. In a floating configuration, the system may resemble an RF antenna-based communication system with a compact radiating element.

Another feature is ability to force an offset between Probe ‘A’ and Probe ‘B’ and scanning for depths abnormalities is no longer static nor fixed. An operator simply adjusts the probe's offset and a new surface area becomes available to testing. That action could take place during the probe's insertion or later, while scanning or stimulating neuro regions.

If two electrodes are being used ‘side-by-side’ during insertion and/or operation they support each other by inner surfaces backing and resting against each other on rails. Insertion of one or two electrodes follows the same process but allows predefined offset between probes position. In order to allow for that action to be successful, the strength and relative flexibility of the structure needs to focus on a vertical strength and live tissue natural resistance against injecting probes.

The dimensions and layout of a carrier wafer used for stacking the electrodes may be changed according to the required application and the electrode layout and dimensions. Specially, while two electrodes are assembled back to back, the two electrodes may perform different measurements. Those cases benefit from neuro probe sensory asymmetry. Two distinct methods of scanning may be available, i.e., one for each electrode.

1. A module of implantable cortical and/or intra-cortical comprising:
a first electrode and a second electrode, each said electrode having
a base with interconnect pads adapted to be connected to a processor,
a stem having interface pads on a first face, the interface pads adapted to contact soft tissue,
routing tracks between the interconnect pads and the interface pads, and
abutment surfaces on the second face of each said electrode;
wherein the abutment surfaces of the first electrode and of the second electrode are complementary, whereby the first electrode and the second electrode form a translation joint along a length of the stem when the electrodes are joined.

2. The module according to claim 1, wherein at least one of the electrodes has multiple shafts for the routing tracks.

3. The module according to claim 1, wherein the routing tracks are embedded in dielectric and/or passivation material.

4. The module according to claim 1, wherein the abutment surfaces of the first electrode form a pair of rails, and the abutment surfaces of the second electrode form shoulders complementary to the rails.

5. The module according to claim 1, wherein the abutment surfaces of the first electrode form a rail, and the abutment surfaces of the second electrode form a groove complementary to the rail.

6. The module according to claim 1, wherein the interface pads are interface stimulation pads and/or recording pads.

7. The module according to claim 1, further comprising at least one metal plate between the first electrode and the second electrode in the translational joint.

8. The module according to claim 7, wherein the at least one metal plate is a RF telecommunication component.

9. The module according to claim 8, wherein the RF telecommunication component operates in one or more of unconnected/floating, GND connected, and VCC connected modes.

10. The module according to claim 1, wherein the second face of the first electrode and/or the second electrode is blank.

11. The module according to claim 1, wherein the stem had a tapered end away from the base.

12. The module according to claim 1, further comprising releasable fastener means to block the translational joint.

13. The module according to claim 12, wherein the releasable fastener means include a mounting hole in the first electrode and/or the second electrode and a screw.

14. The module according to claim 1, wherein the interconnect pads are in an array.

15. The module according to claim 1, wherein the interface pads are in an array.

16. The module according to claim 1, further comprising a motor to actuate the translational joint.

17. The module according to claim 1, wherein the abutment surfaces are in the base.
